(19) United States
(12) Patent Application Publication
(10) Pub. No.: US 2005/0020493 A1
(43) Pub. Date: Jan. 27, 2005

(54) Use of a polyfunctional active substance mixture as an antagonist against harmful substances contained in tobacco smoke

(76) Inventors: Karl Hecht, Berlin (DE); Egon Tech, Rostock (DE); Ronald Demhlow, Jahnsfelde (DE); Ekkard Arbeit, Berlin (DE); Ronald Demhlow, Birkenwerder (DE)

Correspondence Address: BIRCH STEWART KOLASCH & BIRCH, PO BOX 747, FALLS CHURCH, VA, 22040-0747, US

(21) Appl. No.: 10/485,516

(22) PCT Filed: Feb. 15, 2002
(86) PCT No.: PCT/DE02/00563

(30) Foreign Application Priority Data
Feb. 16, 2001 (DE) 101-07-731.9

(51) Int. Cl. A61K 38/17

(52) U.S. Cl. 514/12

(57) ABSTRACT

The invention relates to the use of a polyfunctional active substance mixture with anti-inflammatory, spasmolytic and anti-stress action comprised of a fraction of specific peptides with molar weights of up to 10,000 dalton and/or of a fraction of essential and non-essential amino acids, and the polyfunctional active substance mixture is obtained from a multifactorial immune-modulator mixture serving as an antagonist against the harmful substances contained in tobacco smoke with a health-protecting function during the smoking of tobacco products. It is preferably applied in the form of an inhalant (adhesion principle, atomization) and leads to the amelioration, elimination and prevention of inflammations of the mucous membranes, of regulatory disorders of the organ systems with smooth muscles (blood vessels, bronchial system, esophagus, bladder, stomach, intestines, etc.), of the myocardial function as well as of the central and peripheral nerve systems that are induced by the harmful substances contained in tobacco smoke.

The invention relates to the use of a polyfunctional active substance mixture having anti-inflammatory, spasmolytic and anti-stress action comprised of a fraction of specific peptides with molar weights of up to 10,000 dalton and of a fraction of essential and non-essential amino acids, and whereby the mixture was obtained from a multifactorial immune-modulator mixture and is suitable for use as an antagonist against harmful substances contained in tobacco smoke and having a health-protecting function during the smoking of tobacco.

The mixture is preferably applied as an inhalant (adhesion principle, atomization) and results in the amelioration, elimination and prevention of inflammations induced by the harmful substances contained in tobacco smoke of the mucous membranes, of regulatory disorders of the organ systems with smooth muscles (including blood vessels, bronchial system, esophagus, bladder, stomach, intestines, etc.), of the myocardial function as well as of the central and peripheral nervous system. The polyfunctional active substance mixture is obtained from a multifactorial immune-modulator blend.

The number of scientific works addressing the harmful effects that tobacco smoking has on health and the pathogenic effects of tobacco smoking is endless. This has led to a situation in which governments and courts must now deal with this problem and will continue to have to deal with it. We are presently listing only some of the examples.
1. EU-Commissioner for Health and Consumer Protection, David Byrne, (Focus 11/2000, p. 362/363), “Half a million EU citizens die annually as a consequence of the consumption of tobacco—this is one death per minute.”
2. The World Health Organization is negotiating an anti-tobacco convention (same source).
3. The smoking of pregnant mothers increases prenatal stresses with carcinogenic and teratogenic substances (Deutsches Ärzteblatt [German Physicians' Gazette] 97/39 2000, p. C 1873).
4. In a jury trial in the United States, the US tobacco industry was sentenced to pay $145 million to sick smokers. This is the highest judgment ever achieved for damages in a civil case in the United States (Focus 51/2000, p. 168).
   A Brief Summary of the Essential Pathogenic Consequences of Tobacco Smoking

1. It is known that tobacco smoking, primarily cigarette smoking, has harmful effects due to the harmful substances contained therein which cause chronic inflammations of the mucous membranes of the digestive and respiratory systems as well as of the eyes. Primarily affected are the mucous membranes of the mouth, the nose, the throat and the larynx, the bronchial system and the bronchioles, the esophagus, the stomach, the duodenum, the small intestine and the large intestine. Many people suffer from chronic conjunctivitis due to cigarette smoking. These inflammations that are caused by the harmful substances in tobacco smoke frequently lead to ulcerations in the digestive tract, the complete or partial inhibition of the resorption of vitamins C and beta carotene—vitamin A —(which may cause diseases specific to a vitamin deficiency), infectious diseases of the respiratory organs, etc.; finally, the ultimate consequence of this is a high incidence of health-related costs (refer to litigation in the United States).
2. It is known that the harmful substances contained in tobacco smoke (approximately 200 harmful substances have been detected) result in inflammations of the mucous membranes, disorders of the smooth muscles presenting as contraction or spasm of the blood vessels, in the bronchial system and in the digestive system. Deficient blood supply to the heart (myocardial infarction), to the skin, the brain, the extremities (smoker's leg, etc.), and primarily chest-related complaints of the smoker are the most common disorders, as reflected in the pertinent statistics.
3. It is known that substances contained in tobacco smoke also act as stress factors and, e.g., cause adrenalin, the adrenal medullary hormone, to be increasingly released into the blood stream triggering generalized distress. This causes, on the one hand, the pathological manifestations listed under 1. and 2. to be stimulated and thereby to become enhanced, and triggers or causes, on the other hand, the regulatory disorders that are known consequences of distress, e.g., of the immune system and the transmitter system (including regulatory peptides and neuro-peptides).
4. It is known today that smoking constitutes an addiction and that quitting is usually not only difficult but also accompanied by many severe pathological withdrawal symptoms. This is why many smokers continue to smoke even though they are familiar with the health-related risks.
5. The logical conclusion that is drawn based on these scientific findings is that smoking is harmful to a person's health and that every pack of cigarettes or related advertising must contain a warning of these harmful effects.
6. The tobacco smoker is addicted and intends to continue smoking because the smoker is addicted even though the smoker is aware of the fact that he/she is harming his/her health. This leads to a constant psychological conflict, often also taking place on an unconscious level.
7. A substance that protects against the health-related damage due to tobacco smoking has not been available to date, even though such a substance would be urgently indicated and of great usefulness in order to ease, first, the stress of the internal conflict that the already addicted smoker is struggling with and, second, to protect the already addicted smoker from additional health-related harm of tobacco smoking.

Therefore, the object of the present invention consisted in identifying an antagonist against the harmful substances contained in tobacco smoke that has a health-protecting function and that on its part does not pose a health risk or cause addiction, that does not compromise the taste of the tobacco product and the smoking enjoyment, that is easily usable and that serves to ameliorate, eliminate and prevent tobacco smoke induced
inflammations of the mucous membranes;
regulatory disorders;
the function of the organ systems with smooth muscle (bronchial system, blood vessels, esophagus, bladder, stomach, intestines, etc.);
the cardiac function; and,
the functions of the central and peripheral nerve systems.

It was possible to achieve this object with a polyfunctional active substance mixture that is characterized by its anti-inflammatory, spasmolytic and anti-stress action and that is comprised of a fraction of specific peptides with molar weights of up to 10,000 dalton and/or of a fraction of essential and non-essential amino acids obtained from a multifactorial immune-modulator blend.

In terms of its application, the polyfunctional active substance mixture is preferably incorporated with the tobacco product and administered to the body as an inhalant (adhesion principle, atomization via respiratory pathways and respiratory organs) by way of a mouth tip filter of the tobacco product (cigarette, cigar, cigarillo, tobacco pipe).

Multifactorial immune-modulator mixtures with anti-inflammatory action as such are known in the art. A modulator mixture that is essentially described in DE 195 12 227 C1 is used; but said mixture differs from the blend in DE 195 12 227 C1 in that it represents a fraction of specific peptides with a molecular weight of up to 10,000 dalton and/or of a fraction of essential and non-essential amino acids. It is synthesized analogously to DE 195 12 227 C1, i.e., it is obtained from somatic cells and, in contrast to DE 195 12 227C1, the fraction of specific peptides with a molecular weight of up to 10,000 dalton and/or containing essential and non-essential amino acids is isolated by way of high-speed centrifuging at a running time of 24 hours. This means the polyfunctional active substance mixture with anti-inflammatory, spasmolytic and anti-stress action comprised of a fraction of specific peptides with a relative molar mass of up to 10,000 dalton and/or of essential and non-essential amino acids [is obtained] from the somatic cells by way of
incubation at temperatures that are typical for the original species in the range of 20 to 39° C. for a time period of 2 to 8 days;
followed by lysis;
harvested together with the conservation medium; and,
separation of the components having a molar mass of over 10,000 dalton and simultaneous isolation of the fraction of specific peptides with molar weights of up to 10,000 and/or of the fraction of essential and non-essential amino acids by way of high-speed centrifuging at a running time of 24 hours.
A polyfunctional active substance mixture containing a fraction of specific peptides with a relative molar mass of between 200 and 6,000 dalton is preferred. This mixture was achieved via reconcentration by a factor of 1,000 of the obtained fractions with proteins <10,000 dalton and using physical means. The content materials were analyzed by way of mass spectroscopy.

These peptides <10,000 dalton that are contained in the mixture are peptides that also occur in the human organism, which is why an “endogenic” active substance mixture is available that is well tolerated. Endogenic peptides are widely known and have been described. Within the meaning of the present invention, preferably those peptides are active within the polyfinctional mixture of active substances that are to be described as acting in the way of a homeostasis regulator, stress regulator, as acting upon the smooth muscles and/or as neuro-peptides (Hecht, K., W-E Vogt, E. Wachtel, P. Oehme and M.G. Airapetjanz: Sleep-Regulating Peptides. Beiträge zur Wirkstoffforschung [Contributions to the Active Substance Research]. Vol. 37 dito, 1990), (Oehme, P., H. Bienert, K. Hecht, J. Bergmann: Substance P. Beiträge zur Wirkstoffforschung [Contributions to the Active Substance Research]. Vol. 12 dito, 1981), (Marsan, C. A., W. Z. Trazyk (eds.): Neuropeptides and Neural Transmission. International Brain Research Organization (IBRO) Monograph Series: Volume 7. Raven Press New York), Oehme, P., H. Löwe, E. Göres (eds.): Peptide and Adaptation Basic Research and Clinical Aspects. Beiträge zur Wirkstoffforschung [Contributions to the Active Substance Research], Vol. 36, Academic-Industrial Complex Pharmaceutical Research Berlin (1990), Oehme, P., H. Löwe, E. Göres (eds.): Peptides in the Nervous System. Beiträge zur Wirkstoffforschung [Contributions to the Active Substance Research], Vol. 24 (1985).

A health-protecting antagonist against tobacco smoke was found in this polyfunctional active substance mixture that is preferably introduced as an inhalant (adhesion principle, atomization) by way of a mouth tip filter on the tobacco product (cigarette, cigar, cigarillo, tobacco pipe, etc.) and thereby administered to the body.

For this purpose, the polyfunctional active substance mixture is integrated into the mouth piece (filter) of the tobacco product (cigarette, cigar, cigarillo, tobacco pipe etc.) as part of the manufacturing process in the form of a substrate that is to be atomized, and that is in fact atomized in accordance with the adhesion principle, while smoking; thereby it is blended in with the tobacco smoke and introduced into the body.

The simultaneous application with the tobacco product does in no way compromise the tobacco taste for the smoker. The polyfunctional active substance mixture has moreover no toxically relevant effect. It develops relevance for better health even at very low concentrations (10⁻⁶ to 10⁻⁹ g/kg of body weight), which means it is efficacious via a bioactive trigger function. The polyfunctional active substance mixture has specific effective properties that are to be described as anti-inflammatory, promoting blood flow, stimulating resorption and permeability, relaxing spasms, immuno-modulating, reducing distress, stimulating eustress and regulating homeostasis. The dose-action relationship preferably follows the principle of the hyperbola curve. After a completed bioactive trigger function effect, it is quickly metabolized in the body, which means that overdoses are not possible even in heavy smokers. The development of any resistance vis-a-vis pathogenic organisms was not shown.

The substance leads to an amelioration, elimination and prevention of inflammations of the mucous membranes, of regulatory disorders of the smooth muscle organ systems (blood vessels, bronchial system, esophagus, bladder, stomach, intestines, etc.) of the myocardial function as well as of the central and the peripheral nerve systems that are induced by the harmful substances contained in tobacco smoke.

Using these example, the invention will be illustrated in more detail:

### EMBODIMENTS

1. Synthesis of the Mixture

### EXAMPLE 1

The diploid calf kidney cell line MDBK (ATCC no. CCL 22) is, corresponding to its multiplication rate of 1:4 in DMEM, added to 8% newborn calf serum and then incubated inside roller bottles at 38° C. Larger quantities are, following the adaptation of the cells, more favorably bred inside spinning culture vessels or inside fermenters. After 3 to 4 days the culture has optimally completed its growth phase. The breeding medium is removed and replaced with MEM without serum. The cell cultures are incubated for an additional 5 to 7 days. The longer the incubation time, the more the intracellular pool is deprived of its essential nutrients; mediators and low-molecular metabolic end products are found at increasing concentrations in the MEM. The accumulation of metabolic products and the intracellular lack of essential nutrients is ultimately a stress factor for the aging cell culture. The cell culture reacts to this situation with the synthesis of various “stress proteins.” These stress proteins become concentrated inside the cell. The cell cultures are regularly examined under a light microscope during the concentration phase. No cytopathic changes may be visible after the concentration phase is concluded. The concentration phase is finished with the alkaline purification of the MEM using NaOH and adjustment to a pH 10 as well as a temperature increase to 50° C. for the duration of 60 minutes. Instead of shifting the pH value, freezing of the cells has also proved effective. Freezing occurs favorably at −20° C. After that time, the died-off cells can be easily shaken off the surface of the culture vessel. Temperature increase and pH-shift do not influence the biological activity of the modulator mixture. The conservation medium with the extracellular and intracellular modulators is now freed of cell detritus and particulate components by way of step filtration. The obtained filtrate contains the mixture of modulators, which are freed for the most part of membrane components. Sterile storage without loss of activity is possible for several weeks at 4° C. or by freezing (−20° C.) for at least one year.

By way of ultrafiltration with commonly used instruments, and while the pore size of the filtration membranes holds back globular molecules, fractions with a relative molar mass starting at approximately 10,000 [dalton] are retained. The retained substance contains all extracellular and intracellular materials with a relative molar mass for globular proteins that exceeds 10,000 [dalton] and particles with diameters of less than 9 nm.

The polyfunctional active substance mixture according to the invention comprised of the fraction of specific peptides up to a molar weight of 10,000 dalton and of essential and non-essential amino acids was obtained by ultracentrifuge with a running time of 24 hours from the protein mixture (analogous to DE 195 12 227 C1 of Oct. 3, 1996). The fractions with materials of up to 10,000 dalton molar weight were reconcentrated by a factor of 1,000 and subsequently analyzed by way of mass spectroscopy to determine their content materials. The obtained spectrum documented a particularly high level of materials with molar weights of between 200 and 6,000 dalton.

The polyfunctional active substance mixture that is obtained in this way can be stored without loss of activity for several weeks at 4° C. or frozen (−20° C. and lower) for at least one year. It can also be lyophilized in the usual manner. The purity, harmlessness and efficacy of the modulator mixture according to the invention is verified by way of the corresponding control testing. The purity and harmlessness checks are conducted in accordance with the prescribed methods of the European Pharmacopoeia and/or the German Pharmacopoeia [DAB=Deutsches Arzneimittelbuch] 10. Protein determination and polyacryl gel electrophoresis are conducted using the long known standard protocols.

Purity

1. Testing for sterility in accordance with the European Pharmacopoeia/German Pharmacopoeia 10;
2. Determination of the protein content in accordance with Lowry;
3. Separation and quantification of the main proteins by way of polyacrylamide gel electrophoresis under denaturing conditions.
   Harmlessness
1. Testing for the absence of pyrogene in accordance with the European Pharmacopoeia/German Pharmacopoeia 10;
2. Testing for acute toxicity in accordance with the European Pharmacopoeia/German Pharmacopoeia 10;
3. Testing for teratogeneity in accordance with the European Pharmacopoeia/German Pharmacopoeia 10.

A typical preparation is sterile, free of pyrogene, non-toxic and non-teratogenic. It contains approximately 2 to 3 mg protein per ml.

### EXAMPLE 2

Chicken fibroblasts, obtained by way of typtic cell isolation of chicken embryos, are cultivated in accordance with example 1 in roller bottles. Reconcentration, harvesting and treatment as well as testing for identity, harmlessness, and efficacy was conducted in correspondence with example 1. Achieved are essentially identical results to example 1.

### EXAMPLE 3

A diploid, limited growth tube epithelium cell line of the pig (Latzke, 1993) underwent stationary cultivation in accordance with example 1. Reconcentration, harvesting and treatment as well as testing for identity, harmlessness, and efficacy was conducted in correspondence with example 1.

2. Application Examples

### EXAMPLE 4

In the private practice of a general medical practitioner of alternative medicine, 22 patient volunteers suffering [respectively] from an acute infection of the respiratory pathways were administered the described polyfunctional active substance mixture in the form of an one-time inhalant. Within 24 hours, the symptoms were no longer detectable. In contrast, in patients (n=28) who presented with the same symptoms at the same time and who were treated with the conventional therapeutic means, this disease process lasted 3 to 7 days.

### EXAMPLE 5—SINGLE CASES

5.1 Bronchial pneumonia: A 48-year-old female patient volunteer who had been suffering from bronchitis and an undiagnosed bronchial pneumonia was treated with the described multifunctional active substance mixture in the form of an inhalant administered twice daily. As early as after two inhalations, the symptoms started to subside. Within a week, the symptoms had for the most part disappeared.
5.2 Viral bronchitis: A 76-year-old male patient had been suffering for approximately 2 weeks from a therapy-resistant chronic viral bronchitis accompanied by strong nightly attacks of a hacking, dry cough. After the first inhalation of the described polyfunctional n active substance mixture, the nightly hacking cough eased up; after two inhalations the entire bronchitis disappeared.
5.3: A 76-year-old male patient had been suffering for years from frequently occurring nose bleeds (apparently of an allergic nature). After eight inhalations of the described polyfunctional n active substance mixture administered twice daily, the nose bleeds did not return. The follow-up control period is currently 6 months.

### EXAMPLE 6—HYPERFUNCTION SMOKING, ACTIVE SUBSTANCE INHALATION

The efficacy of the described polyfunctional active substance mixture was tested in 12 volunteers while they were each smoking a cigarette using a very sensitive heart function warning system that operated on ECG basis (Jumatow Moskow). One time the cigarette was smoked without the polyfunctional active substance mixture and one time with the mixture. While the heart function warning system signaled the risk when the cigarettes without active substance inhalant were smoked, the warning was not triggered when the atomized active substance mixture was inhaled simultaneously with the cigarette smoke.

1. Use of a polyfunctional active substance mixture with anti-inflammatory, spasmolytic and anti-stress action as an antagonist against the harmful substances contained in tobacco smoke comprised of a fraction of specific peptides with molar weights of up to 10,000 dalton and/or of a fraction of essential and non-essential amino acids, and said polyfunctional active substance mixture was obtained from a multifactorial immune-modulator mixture:
by way of incubation at the temperatures that are typical for the original species of 20 to 39° C. for a time period of 2 to 8 days;
followed by lysis;
harvested together with the conservation medium; and,
separation of cell components which have a molar mass of over 10,000 dalton by way of high-speed centrifuging at a running time of 24 hours; and,
gaining the fraction of specific peptides with molar weights of up to 10,000 dalton and/or of the fraction of essential and non-essential amino acids.

2. Use as claimed in claim 1 wherein a fraction of specific peptides with molar weights of between 200 and 6,000 dalton and/or a fraction of essential and non-essential amino acids are applied.

3. Use as claimed in claim 1 wherein mixtures of peptides, as well as neuro-peptides contained therein, are applied that regulate homeostasis, act upon the smooth muscles and regulate stress.

4. Use as claimed in one of the claims 1 to 3 wherein the mixture is applied at low concentrations, preferably at a dosage of 10⁻⁶ to 10⁻⁹ g/kg of body weight and wherein its action occurs primarily in the form of a bioactive triggering function.

5. Use as claimed in one of the claims 1 to 4 wherein the dose-action relationship follows the principle of a hyperbolic curve whereby an overdose is impossible.

6. Use as claimed in one of the claims 1 to 5 wherein the mixture is applied in a specific filter (specific mouth tip) of the tobacco product (cigarette, cigar, cigarillo, tobacco pipe, etc.) and wherein the mixture is incorporated into the tobacco product at the time of the manufacture of the tobacco product and inhaled during smoking.

7. Use as claimed in one of the claims 1 to 6 wherein it [the filter] itself is not harmful to the smoker's health, nor does it constitute a risk for addiction, and it does not change the smoking enjoyment.

8. Use as claimed in one of the claims 1 to 7 wherein the polyfunctional active substance mixture is applied for ameliorating, eliminating and preventing:
inflammations of the mucous membranes;
regulatory disorders;
[impairment] of the function of the organ systems with smooth muscles (bronchial system, blood vessels, esophagus, bladder, stomach, intestines, etc.);
[impairment] of the myocardial functions; and,
[impairment] of the functions of the central and peripheral nerve system induced by the harmful substances contained in tobacco smoke.

9. Specific filters for tobacco products wherein these filters comprise a polyfunctional active substance mixture as claimed in one of the claims 1 to 4 featuring a health-protecting function during the smoking of tobacco.
